(19) United States
(12) Patent Application Publication
(10) Pub. No.: US 2026/0158058 A1
(43) Pub. Date: Jun. 11, 2026

(54) METHOD AND PRODUCT FOR TREATING ACNE, AND METHOD FOR PREPARING THE PRODUCT

(71) Applicants: AITUSHENG (SHANGHAI) MEDICAL TECHNOLOGY CO., LTD., Shanghai (CN); ZIBO AITUSHENG MEDICAL TECHNOLOGY CO., LTD., Zibo (CN)

(72) Inventors: Liangfang WANG, Shanghai (CN); Kui ZHAO, Shanghai (CN); Kai WANG, Shanghai (CN)

(21) Appl. No.: 19/537,572

(22) Filed: Feb. 12, 2026

Related U.S. Application Data
(63) Continuation of application No. PCT/CN2025/128527, filed on Oct. 17, 2025.

(30) Foreign Application Priority Data
Oct. 21, 2024 (CN) 202411471222.1

(51) Int. Cl. A61K 33/00; A61K 9/08; A61P 17/10

(52) U.S. Cl. CPC A61K 33/00; A61K 9/08; A61P 17/10

(57) ABSTRACT

Provided are a method for treating acne, a method for preventing and/or treating sebaceous gland homeostasis imbalance, a product for treating acne or preventing and/or treating sebaceous gland homeostasis imbalance and a method for preparing the same. The method for treating acne includes administering an aqueous silicic acid solution in a stable state to a subject in need thereof. The method for preventing and/or treating sebaceous gland homeostasis imbalance includes administering an aqueous silicic acid solution in a stable state to a subject in need thereof. The product for treating acne or preventing and/or treating sebaceous gland homeostasis imbalance includes an aqueous silicic acid solution in a stable state as an active ingredient. A mass concentration of silicic acid in the aqueous silicic acid solution in a stable state ranges from 0.5% to 2%.

### CROSS-REFERENCE TO RELATED APPLICATIONS

The present application is a continuation of International Application No. PCT/CN 2025/128527, filed on Oct. 17, 2025, which claims priority and benefits of Chinese Patent Application No. 202411471222.1, filed with the China National Intellectual Property Administration on Oct. 21, 2024. The disclosures of the aforementioned applications are herein incorporated by reference in their entireties.

### FIELD

The present disclosure relates to the field of biomedicine technologies, and in particular, to use of an aqueous silicic acid solution in a stable state in the preparation of a product for treating acne; and more particularly, to use of an aqueous silicic acid solution in a stable state in the preparation of a product for preventing and/or treating sebaceous gland homeostasis imbalance, and a product and a method for treating acne or preventing and/or treating sebaceous gland homeostasis imbalance.

### BACKGROUND

Acne is a chronic inflammatory skin disease occurring in pilosebaceous units. In the early stage, the acne mostly manifests as comedones and inflammatory papules. As the disease progresses, inflammation develops, manifesting as pustules and nodules, and even irreversible scars may appear. With a long course, recurrent attacks of the disease may leave hyperpigmentation or scars. The acne predominantly occurs in body parts with abundant sebum secretion, such as the face, chest, and back, which causes a serious impact on patients' physiology, psychology, and social interaction, and may lead to depression or even suicide. The acne is one of the most common disfiguring skin diseases in dermatology. Studies have shown that excessive sebum secretion and changes in lipid composition are the core of the occurrence and development of acne, running through the entire course of the disease. The sebaceous gland is a holocrine gland. Androgens may stimulate proliferation and differentiation of new sebaceous gland cells, promote lipid synthesis, and move the lipids to a center of the gland to form small lipid droplets. When a content of lipid droplets in the cytoplasm reaches a certain level, the sebaceous gland cells will disintegrate spontaneously and release lipids.

At present, in clinical treatment of acne, retinoids are the main medicaments for sebaceous gland homeostasis imbalance. Although the retinoids have a good curative effect in inhibiting sebum secretion, long-term systemic application of such medicaments may cause many adverse reactions, such as liver damage, intestinal mucosal damage, cardiovascular diseases, teratogenicity, and mental and psychological problems.

### SUMMARY

A first aspect of the present disclosure provides a method for treating acne. The method comprises administering an aqueous silicic acid solution in a stable state to a subject in need thereof. A mass concentration of silicic acid in the aqueous silicic acid solution in a stable state ranges from 0.5% to 2%.

A second aspect of the present disclosure provides a method for preventing and/or treating sebaceous gland homeostasis imbalance. The method comprises administering an aqueous silicic acid solution in a stable state to a subject in need thereof. A mass concentration of silicic acid in the aqueous silicic acid solution in a stable state ranges from 0.5% to 2%.

A third aspect of the present disclosure provides a product for treating acne or preventing and/or treating sebaceous gland homeostasis imbalance. The product comprises an aqueous silicic acid solution in a stable state as an active ingredient. A mass concentration of silicic acid in the aqueous silicic acid solution in a stable state ranges from 0.5% to 2%.

A fourth aspect of the present disclosure provides a method for preparing the product according to the third aspect of the present disclosure. The method comprises mixing the aqueous silicic acid solution in a stable state with pharmaceutically acceptable excipients.

### BRIEF DESCRIPTION OF THE DRAWINGS

FIG. 1 shows an appearance photograph of sebaceous gland patches in golden hamsters of each group in Example 1.

FIG. 2 shows an appearance size of sebaceous gland patches in golden hamsters of each group in Example 1.

FIG. 3 shows a histopathological manifestation of sebaceous gland patches in each group after administration in Example 1.

### DETAILED DESCRIPTION

Embodiments of the present disclosure will be described in detail below. The embodiments described below are illustrative only, and are intended to explain, rather than limiting, the present disclosure.

It should be noted that the terms “first” and “second” are only used for descriptive purposes, and cannot be understood as indicating or implying relative importance or implicitly indicating the number of indicated technical features. Therefore, the features associated with “first” and “second” may explicitly or implicitly include at least one of the features. Further, in the description of the present disclosure, “plurality of” means at least two, unless otherwise specifically defined.

To facilitate a better understanding of the present disclosure, certain technical and scientific terms are specifically defined below. Except where clearly defined otherwise elsewhere in the present disclosure, all other technical and scientific terms used in the present disclosure have the meanings commonly understood by those of ordinary skill in the art to which the present disclosure belongs.

In the present disclosure, the term “comprises” or “includes” is an open-ended expression, meaning including the content specified in the present disclosure, but not excluding content from other aspects.

As used herein, the term “pharmaceutically acceptable excipients” may include any solvents, solid excipients, diluents, or other liquid excipients, etc., suitable for a specific target dosage form. Their use is also contemplated by the present disclosure, except to the extent that any conventional excipients are incompatible with the compounds of the present disclosure, such as any adverse biological effects produced or interactions produced in a harmful manner with any other components of the pharmaceutically acceptable composition.

As used herein, the term “treatment” refers to achieving a desired pharmacological and/or physiological effect. The effect may be prophylactic in terms of completely or partially preventing the disease or its symptoms, and/or therapeutic in terms of partially or completely curing the disease and/or adverse effects caused by the disease. “Treatment” as used herein encompasses the treatment of diseases in mammals, particularly humans, including: (a) preventing the occurrence of the disease or condition in an individual who is susceptible to the disease but has not yet been diagnosed with it; (b) inhibiting the disease, such as halting the development of the disease; or (c) alleviating the disease, such as reducing the symptoms associated with the disease. “Treatment” as used herein encompasses any administration of a medicament or compound to an individual to treat, cure, alleviate, improve, reduce, or inhibit the individual's disease, including but not limited to administering a medicament containing the compound described herein to an individual in need thereof.

As used herein, the term “administration” refers to introducing a predetermined amount of a substance into a patient by a suitable method. The aqueous silicic acid solution in a stable state of the present disclosure can be administered via any common route as long as it can reach the intended tissue. Various modes of administration are contemplated, including intraperitoneal, intravenous injection, intramuscular injection, subcutaneous injection, etc., but the present disclosure is not limited to these exemplified modes of administration. Preferably, the composition of the present disclosure is administered by intravenous injection or subcutaneous injection.

The purpose of the present disclosure is to address a problem existing in a medicament for sebaceous gland homeostasis imbalance, propose an alternative medicament, and use an aqueous silicic acid solution in a stable state to prepare a product for treating acne, to achieve a better curative effect.

To achieve the above objective, the present disclosure provides use of an aqueous silicic acid solution in a stable state in the preparation of a product for treating acne, use of an aqueous silicic acid solution in a stable state in the preparation of a product for preventing and/or treating sebaceous gland homeostasis imbalance, a product for treating acne or preventing and/or treating sebaceous gland homeostasis imbalance, and a method for preparing the product as described according to the third aspect of the present disclosure, which will be described in detail respectively below.

### Use

A first aspect of the present disclosure provides the use of an aqueous silicic acid solution in a stable state in the preparation of a product for treating acne. A mass concentration of silicic acid in the aqueous silicic acid solution in a stable state ranges from 0.5% to 2%. For example, the mass concentration of silicic acid may be 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, or a range between any two of these values such as 0.6% to 2%, 0.7% to 2%. As an active ingredient, the aqueous silicic acid solution in a stable state of the present disclosure has anti-acne efficacy, where a mass concentration of silicic acid in the aqueous silicic acid solution in a stable state ranges from 0.5% to 2%. Meanwhile, the raw material of the aqueous silicic acid solution in a stable state has a low cost and is environmentally friendly and safe. As a result, the aqueous silicic acid solution in a stable state can be used as an alternative medicament with good curative effect compared to traditional retinoids.

It should be noted that the aqueous silicic acid solution in a stable state in the present disclosure is an aqueous solution of silicic acid maintained in a stable state. The aqueous silicic acid solution in a stable state refers to a solution in a stable state without polymerization, and the aqueous silicic acid solution includes at least one of orthosilicic acid (H₄SiO₄), metasilicic acid (H₂SiO₃), or hydrogen silicate ions (HSiO₃⁻). The mass concentration of silicic acid ranging from 0.5% to 2% in the present disclosure refers to a total concentration of the main components ranging from 0.5% to 2%.

### Use

A second aspect of the present disclosure provides the use of the aqueous silicic acid solution in a stable state in the preparation of a product for preventing and/or treating sebaceous gland homeostasis imbalance. A mass concentration of silicic acid in the aqueous silicic acid solution in a stable state ranges from 0.5% to 2%. For example, the mass concentration of silicic acid may be 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, or a range between any two of these values such as 0.6% to 2%, 0.7% to 2%. As an active ingredient, the aqueous silicic acid solution in a stable state of the present disclosure can reduce an area of sebaceous gland patches in golden hamsters and lighten the color of sebaceous gland patches in golden hamsters, where a mass concentration of silicic acid in the aqueous silicic acid solution in a stable state ranges from 0.5% to 2%. Meanwhile, the raw material of the aqueous silicic acid solution in a stable state has a low cost and is environmentally friendly and safe. As a result, the aqueous silicic acid solution in a stable state can be used as an alternative medicament with good curative effect compared to traditional retinoids.

In some embodiments, the aqueous silicic acid solution in a stable state is dispersed as an active ingredient in a matrix of an ointment for external application.

It should be explained that since acne is essentially a manifestation of sebaceous gland homeostasis imbalance, the aqueous silicic acid solution in a stable state of the present disclosure may also be used in the preparation of the product for preventing and/or treating sebaceous gland homeostasis imbalance. The mass concentration of silicic acid in the aqueous silicic acid solution in a stable state ranges from 0.5% to 2%.

### Product

A third aspect of the present disclosure provides a product for treating acne or preventing and/or treating sebaceous gland homeostasis imbalance. According to an embodiment of the present disclosure, the product includes an aqueous silicic acid solution in a stable state as an active ingredient, where a mass concentration of silicic acid in the aqueous silicic acid solution in a stable state ranges from 0.5% to 2%. The mass concentration of silicic acid may be, for example, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, or a range between any two of these values such as 0.6% to 2%, 0.7% to 2%. For the product according to the embodiment of the present disclosure, the aqueous silicic acid solution in a stable state as the active ingredient, can reduce an area of sebaceous gland patches in golden hamsters and lighten the color of sebaceous gland patches in golden hamsters, thereby possessing anti-acne efficacy, where a mass concentration of silicic acid in the aqueous silicic acid solution in a stable state ranges from 0.5% to 2%. Meanwhile, the raw material of the aqueous silicic acid solution in a stable state has a low cost and is environmentally friendly and safe. As a result, the aqueous silicic acid solution in a stable state can be used as an alternative medicament with good curative effect compared to traditional retinoids.

In some embodiments, the product is a cosmetic, a medical device, or a medicament.

In some embodiments, the product is an external application preparation.

In some embodiments, the aqueous silicic acid solution in a stable state includes at least one of orthosilicic acid, metasilicic acid, or hydrogen silicate ions.

### Method

In a fourth aspect of the present disclosure, the present disclosure provides a method for preparing the product as described according to the third aspect of the present disclosure. According to an embodiment of the present disclosure, the method includes mixing the aqueous silicic acid solution in a stable state with pharmaceutically acceptable excipients.

In some embodiments, the mass concentration of silicic acid in the aqueous silicic acid solution in a stable state ranges from 0.5% to 2%. For example, the mass concentration of silicic acid may be 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, or a range between any two of these values such as 0.6% to 2%, 0.7% to 2%.

The solutions of the present disclosure will be explained below in conjunction with the following Example. Those skilled in the art will understand that the following Example is only used to illustrate the present disclosure and should not be regarded as limiting the scope of the present disclosure. For unspecified specific technologies or conditions in the following Example, the technologies or conditions described in the literature in the field or according to the product specifications shall be followed. The reagents or instruments without indicating the manufacturer are conventional products that can be purchased commercially.

### Example 1

To illustrate an effect of use of an aqueous silicic acid solution in a stable state in the preparation of a product for treating acne, this example takes sebaceous gland patches of golden hamsters to simulate human acne for explanation.

Golden hamsters, also known as Syrian hamsters, has dorsal sebaceous gland patches are mainly composed of hair follicles, sebaceous glands, and melanin aggregates, and are androgen-dependent organs. The golden hamsters are similar to humans in anatomical structure and physiological response to hormones, making them an ideal animal model for studying abnormal sebum metabolism in acne. Therefore, the golden hamsters (Syrian hamsters) are often used as animal models for skin pathology research, especially in the field of acne (pimples) research. Sebaceous gland patches refer to small lumps formed when sebaceous glands are overactive, are particularly common on the lateral abdomen of golden hamsters, and may be used to simulate the pathological process of human acne.

Experimental animals: Aqueous silicic acid solutions in a stable state with concentrations of the silicic acid of 0.5%, 1%, and 2% used as active ingredients, which were dispersed in a matrix of a commercial oil-in-water medical ointment as test medicaments for easy use; retinoic acid; and 30 SPF-grade, male, 6 to 7 week-old golden hamsters with a body weight of 110±10 g. The 30 golden hamsters were housed in separate cages in an SPF-grade animal room with a constant temperature of 22±2° C. and a 12-hour light/dark cycle, and fed with special feed for golden hamsters.

Experimental grouping: After 10 days of adaptive feeding, experiments were performed on the 30 golden hamsters. The animals were grouped into 6 groups in total, 5 per group, according to a random number method: a blank group, a matrix group, a 0.5% test medicament, a 1.0% test medicament, a 2.0% test medicament, and a positive control group (0.1% retinoic acid cream). Each group was housed in two cages.

Modeling and detection indexes: Measurement of sebaceous gland patch area. On day 0 of administration, a size of each of dorsal sebaceous gland patches of the golden hamsters was measured with a vernier caliper under natural light, expressed as a maximum transverse diameter×a maximum longitudinal diameter, and an area of each of the sebaceous gland patches was calculated. The size of each of the sebaceous gland patches was measured every 3 days and photographed for archiving.

Pathological examination: Observation of histopathological changes of sebaceous gland patches by HE staining of skin tissue. On day 25 of administration, a skin tissue of dorsal sebaceous gland patches of the golden hamsters was taken, fixed in 4% formaldehyde for 24 hours, then subjected to tissue dehydration, paraffin embedding, paraffin sectioning, and HE staining to observe a thickness, area, number of glandular lobes, number of overlapping layers of the sebaceous glands, and a looseness degree of the sebaceous glands. Operation steps of the HE staining are described below.

In step (1) of dehydration, the fixed skin tissue was placed in a dehydration box and sequentially immersed in ethanol with gradients from low to high (75% ethanol, 90% ethanol, 90% ethanol, 100% ethanol I, 100% ethanol II, 100% ethanol III) for 1 hour each.

In step (2) of clearing, the skin tissue was cleared in Xylene I, Xylene II, Xylene III for 45 minutes each.

In step (3) of paraffin impregnation, the skin tissue was impregnated in Paraffin I, Paraffin II, Paraffin III for 1 hour each.

In step (4) of embedding, after the skin tissue was impregnated with paraffin, it was placed vertically in an embedding frame, and liquid paraffin was continued to be slowly poured into the frame. After the paraffin solidified, the embedded paraffin block was taken out, trimmed, and labeled.

In step (5) of sectioning, excess paraffin was trimmed from a surface of the paraffin block, and the tissue was placed in a microtome when it was about to be exposed. After sectioning into 5 μm sections, the specimens were gently placed in warm water at 42° C. to flatten, then mounted onto a glass slide, and dried at 60° C.

In step (6) of deparaffinization and hydration, the sections were placed in an oven at 60° C. and dried for 1 hour, then immersed in Xylene I and Xylene II for 15 minutes each, followed by 100% alcohol, 95% alcohol, 90% alcohol, 80% alcohol, and 70% alcohol for 5 minutes each, and finally in distilled water for 3 minutes.

In step (7) of staining, the sections were stained with hematoxylin stain for 5 minutes.

In step (8) of water washing, the sections were rinsed with running tap water for about 15 minutes (or placed in alkaline water) to turn blue. However, it should be noted that the water flow should not be too strong to prevent the sections from falling off.

In step (9) of differentiation, the sections were placed in 1% hydrochloric acid ethanol solution to fade for about 2 seconds until they turned red and the color was light enough.

In step (10) of rinsing, the sections were replaced in the running tap water to restore their blue color.

In step (11) of dehydration I, the sections were sequentially immersed in 50% ethanol, 70% ethanol, 80% ethanol for 3 minutes to 5 minutes each.

In step (12) of counterstaining, the sections were counterstained with 0.5% eosin ethanol solution for 1 min.

In step (13) of dehydration II, the sections were washed in 95% ethanol to remove excess red color, then transferred to absolute ethanol for 3 minutes to 5 minutes. Finally, excess ethanol was blotted dry with absorbent paper.

In step (14) of clearing, the sections were placed in Xylene I and Xylene II for 3 minutes to 5 minutes each.

In step (15) of mounting, the sections were mounted with neutral gum.

In this Example, the golden hamsters were used to explore effects of test medicaments with different concentrations on the sebaceous gland patches, and the following results were obtained.

External application of the test medicaments with different concentrations can reduce the area of sebaceous gland patches in the golden hamsters and lighten the color of the sebaceous gland patches.

During the experiment, no significant abnormalities were observed in living conditions of the golden hamsters in all groups, with no obvious adverse reactions systemically or locally. As the administration time prolonged, the sebaceous gland patches in the blank group and matrix group showed dark black color and increased area.

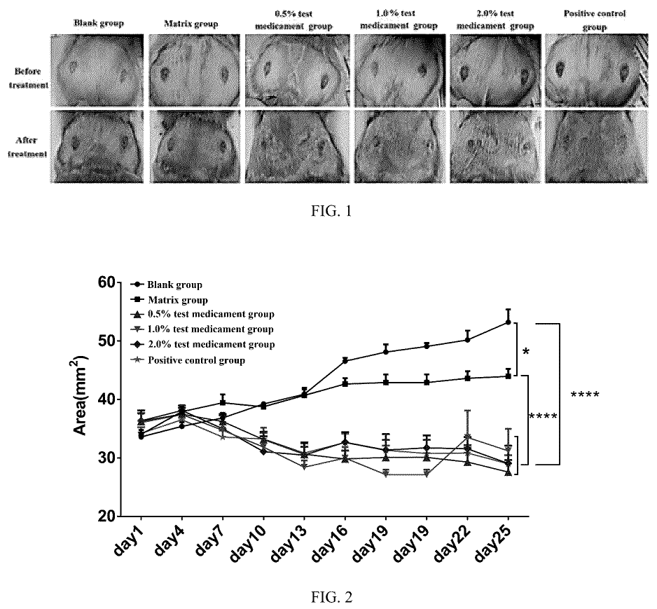

Compared with the golden hamsters in the blank group and matrix group, the sebaceous gland patches of golden hamsters in the test medicament groups with different concentrations and the positive control group showed varying degrees of lightened color and reduced area. However, as shown in FIG. 1 and FIG. 2, there were no significant differences among the test medicament groups with different concentrations, nor between these groups and the positive control group.

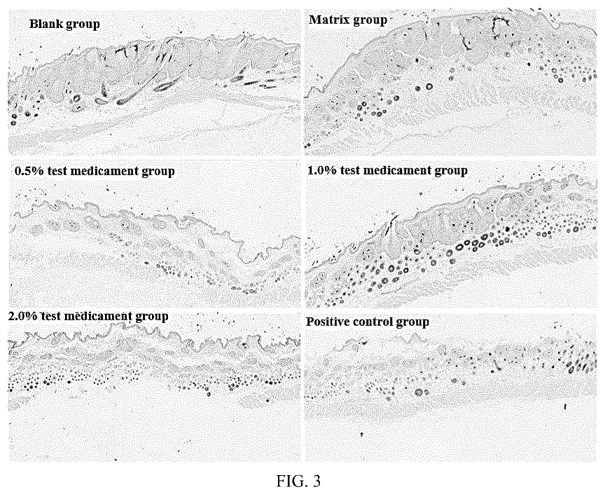

The histopathology of sebaceous gland patches in golden hamsters from different experimental groups was observed. Under the microscope after HE staining, the sebaceous glands in the blank group showed an intact structure, a large number of sebaceous gland lobules distributed in a lobular pattern, large and plump lobules with a plurality of overlapping lobule layers (mostly 3 layers) and dense arrangement. Compared with the blank group, there was no significant change in the sebaceous gland structure of the matrix group. Compared with the blank group, in the 0.5% test medicament group, 1.0% test medicament group, and 2.0% test medicament group, the number of glands and the number of overlapping lobule layers were relatively reduced, and the glands were arranged relatively loosely, which were similar to the results of the positive control group, as shown in FIG. 3, and thus the test medicaments have the potential to be an alternative medicament with good efficacy compared to the traditional retinoids.

Through animal experiments, the effects of the test medicaments with different concentrations on the golden hamster model of cutaneous acne (acne vulgaris, a chronic inflammatory skin disease of the pilosebaceous unit) showed that: as the active ingredient, the aqueous silicic acid solution in a stable state can reduce the area and lighten the color of the sebaceous gland patches in the golden hamsters, thereby possessing the anti-acne efficacy, where a mass concentration of silicic acid in the aqueous silicic acid solution in a stable state ranges from 0.5% to 2%.

In the description of this specification, descriptions with reference to the terms “an embodiment”, “some embodiments”, “examples”, “specific examples”, or “some examples” etc., mean that specific features, structure, materials or characteristics described in conjunction with the embodiment or example are included in at least one embodiment or example of the present disclosure. In this specification, the schematic representations of the above terms do not necessarily refer to the same embodiment or example. Moreover, the described specific features, structures, materials or characteristics may be combined in any one or more embodiments or examples in a suitable manner. In addition, those skilled in the art can combine the different embodiments or examples and the features of the different embodiments or examples described in this specification without contradicting each other.

Although the embodiments of the present disclosure have been shown and described above, it can be understood that the above-mentioned embodiments are exemplary and should not be construed as limiting the present disclosure. Those of ordinary skill in the art can make changes, modifications, substitutions and modifications to the above-mentioned embodiments within the scope of the present disclosure.

What is claimed is:

1. A method for treating acne, the method comprising:
administering an aqueous silicic acid solution in a stable state to a subject in need thereof, wherein a mass concentration of silicic acid in the aqueous silicic acid solution in a stable state ranges from 0.5% to 2%.

2. The method according to claim 1, wherein the aqueous silicic acid solution in a stable state is dispersed as an active ingredient in a matrix of an ointment for external application.

3. The method according to claim 1, wherein the aqueous silicic acid solution in a stable state comprises at least one of orthosilicic acid, metasilicic acid, or hydrogen silicate ions.

4. A method for preventing and/or treating sebaceous gland homeostasis imbalance, the method comprising:
administering an aqueous silicic acid solution in a stable state to a subject in need thereof, wherein a mass concentration of silicic acid in the aqueous silicic acid solution in a stable state ranges from 0.5% to 2%.

5. The method according to claim 4, wherein the aqueous silicic acid solution in a stable state is dispersed as an active ingredient in a matrix of an ointment for external application.

6. The method according to claim 4, wherein the aqueous silicic acid solution in a stable state comprises at least one of orthosilicic acid, metasilicic acid, or hydrogen silicate ions.

7. A product for treating acne or preventing and/or treating sebaceous gland homeostasis imbalance, wherein the product comprises an aqueous silicic acid solution in a stable state as an active ingredient, wherein a mass concentration of silicic acid in the aqueous silicic acid solution in a stable state ranges from 0.5% to 2%.

8. The product according to claim 7, wherein the product is a cosmetic, a medical device, or a medicament.

9. The product according to claim 7, wherein the product is an external application preparation.

10. The product according to claim 8, wherein the product is an external application preparation.

11. The product according to claim 9, wherein the external application preparation is an ointment, a cream, a gel, or a patch.

12. The product according to claim 10, wherein the external application preparation is an ointment, a cream, a gel, or a patch.

13. The product according to claim 7, wherein the aqueous silicic acid solution in a stable state comprises at least one of orthosilicic acid, metasilicic acid, or hydrogen silicate ions.

14. A method for preparing the product according to claim 7, the method comprising:
mixing the aqueous silicic acid solution in a stable state with pharmaceutically acceptable excipients.

15. The method according to claim 14, wherein a mass concentration of silicic acid in the aqueous silicic acid solution in a stable state ranges from 0.5% to 2%.
